(19) United States
(12) Patent Application Publication
(10) Pub. No.: US 2020/0297635 A1
(43) Pub. Date: Sep. 24, 2020

(54) EMBOLIC MICROSPHERES AND METHODS

(71) Applicants: BIOCOMPATIBLES UK LIMITED, Surrey (GB); The Johns Hopkins University, Baltimore, MD (US)

(72) Inventors: Matthew R. Dreher, West Conshohocken, PA (US); Clifford Weiss, Baltimore, MD (US)

(21) Appl. No.: 16/826,577

(22) Filed: Mar. 23, 2020

Related U.S. Application Data
(60) Provisional application No. 62/822,319, filed on Mar. 22, 2019.

(51) Int. Cl. A61K 9/16

(52) U.S. Cl. CPC A61K 9/1635; A61L 29/16

(57) ABSTRACT

This present disclosure relates to compositions and methods useful in therapeutic embolisation and particularly in methods for bariatric arterial embolisation (BAE).

### RELATED APPLICATION

This application claims the benefit of Provisional Application Ser. No. 62/822,319, filed Mar. 22, 2019, the disclosure of which is herein incorporated herein by reference in its entirety.

### FIELD

This present disclosure relates to compositions and methods useful in therapeutic embolisation and particularly in methods for bariatric arterial embolisation (BAE).

### BACKGROUND

Therapeutic embolisation is a minimally invasive procedure in which a material is introduced into a blood vessel by the trans catheter route, in order to occlude the vessel and thus slow or stop blood flow leading to ischemia in the supplied tissue. This approach has been used for some time in the treatment of hyper-vascular tumours such as hepatocellular carcinoma, and for the treatment of benign growths such as uterine fibroids. Recent pre-clinical observations have suggested that embolisation of blood vessels supplying the gastric fundus (known as bariatric arterial embolisation or BAE) may be useful in the control of weight gain.

### SUMMARY

In some aspects, the present disclosure provides compositions that comprise a population of polymeric microspheres that comprise a polymer and have a native size distribution in which not more than 10% of the microspheres have a diameter of less than 120 μm and not more than 10% of the microspheres have a diameter greater than 200 μm.

In some embodiments, which can be used in conjunction with the preceding aspects, the microspheres may have a mean compression modulus of greater than 1000 kPa.

In some embodiments, which can be used in conjunction with the preceding aspects and embodiments, the microspheres have a mean compression modulus of at least 5 times that of Bead Block® 300-500.

In some embodiments, which can be used in conjunction with the preceding aspects and embodiments, the microspheres have a native size distribution in which not more than 5% of the microspheres have a diameter less than 100 μm and not more than 5% of the microspheres have a diameter greater than 200 μm.

In some embodiments, which can be used in conjunction with the preceding aspects and embodiments, the microspheres have a native size distribution such that not more than 5% of the microspheres have a diameter less than 120 μm and not more than 10% of the microspheres have a diameter greater than 185 μm.

In some embodiments, which can be used in conjunction with the preceding aspects and embodiments, not more than 10% of the microspheres have a penetration value, in a swine kidney model, of less than 80 μm.

In some embodiments, which can be used in conjunction with the preceding aspects and embodiments, not more than 10% of the microspheres have a penetration value of greater than 300 μm.

In some embodiments, which can be used in conjunction with the preceding aspects and embodiments, not more than 5% of the microspheres have a penetration value of less than 80 μm and not more than 5% of the microspheres have a penetration value of greater than 300 μm.

In some embodiments, which can be used in conjunction with the preceding aspects and embodiments, not more than 5% have a penetration value of less than 90 μm and not more than 5% of the microspheres have a penetration value of greater than 250 μm.

Other aspects of the present disclosure pertain to compositions that comprise a population of polymeric microspheres, which comprise a polymer and in which not more than 10% of the microspheres have a penetration value, in a swine kidney model, of less than 80 μm.

In some embodiments, which can be used in conjunction with the preceding aspects, not more than 10% of the microspheres have a penetration value of greater than 300 μm.

In some embodiments, which can be used in conjunction with the preceding aspects and embodiments, not more than 5% of the microspheres have a penetration value of less than 80 μm and not more than 5% of the microspheres have a penetration value of greater than 300 μm.

In some embodiments, which can be used in conjunction with the preceding aspects and embodiments, not more than 5% have a penetration value of less than 90 μm and not more than 5% of the microspheres have a penetration value of greater than 250 μm.

In some embodiments, which can be used in conjunction with the preceding aspects and embodiments, the microspheres have a native size distribution in which not more than 10% of the microspheres have a diameter of less than 120 μm and not more than 10% of the microspheres have a diameter greater than 200 μm.

In some embodiments, which can be used in conjunction with the preceding aspects and embodiments, the microspheres have a native size distribution such that not more than 5% of the microspheres have a diameter less than 120 μm and not more than 10% of the microspheres have a diameter greater than 185 μm.

In some embodiments, which can be used in conjunction with the preceding aspects and embodiments, the microspheres have a mean compression modulus of greater than 1000 kPa.

In some embodiments, which can be used in conjunction with the preceding aspects and embodiments, the microspheres have a mean compression modulus of at least 10 times that of Beadblock® 300-500.

In some embodiments, which can be used in conjunction with the preceding aspects and embodiments, the polymer is a hydrogel.

In some embodiments, which can be used in conjunction with the preceding aspects and embodiments, the polymer comprises poly vinyl alcohol.

In some embodiments, which can be used in conjunction with the preceding aspects and embodiments, the polymer is imageable.

In some embodiments, which can be used in conjunction with the preceding aspects and embodiments, the polymer is radiopaque.

In some embodiments, which can be used in conjunction with the preceding aspects and embodiments, the polymer comprises between 70 and 150 mg of iodine per mL of settled microspheres covalently bound to the polymer, preferably 85-120 mg/mL and particularly 90-110 mg/mL of settled microspheres.

Other aspects of the present disclosure pertain to pharmaceutical compositions that comprise a population of polymeric microspheres according to any preceding aspects and embodiments and a pharmaceutically acceptable diluent.

Other aspects of the present disclosure pertain to methods of inducing weight loss or of slowing weight gain in a subject in need thereof, comprising delivering to the capillary bed of the gastric fundus of the subject, an effective amount of a population of microspheres according to any of the above aspects and embodiments or of a pharmaceutical composition according to any of the above aspects and embodiments.

Other aspects of the present disclosure pertain to methods for the treatment of obesity in a subject in need thereof, comprising delivering to the capillary bed of the gastric fundus of the subject, an effective amount of a population of microspheres according to any of the above aspects and embodiments or of a pharmaceutical composition according to any of the above aspects and embodiments.

In some embodiments, the microspheres are delivered to the subject by the transcatheter route.

Other aspects of the present disclosure pertain to compositions according to any of the above aspects and embodiments for use in a method of inducing weight loss or of slowing weight gain in a subject in need thereof.

### BRIEF DESCRIPTION OF THE DRAWINGS

FIG. 1 is a size distribution graph illustrating the native size distributions of a number of commercially available microsphere preparations, in comparison to test samples.

FIG. 2 is a size distribution histogram of the penetration values for radiopaque 102 microspheres in swine kidney (iodine 129 mg/ml).

FIG. 3 is a size distribution histogram of the penetration values for radiopaque 304 microspheres in swine kidney (iodine 113 mg/ml).

FIG. 4 is a size distribution histogram of the penetration values for (non radiopaque) Bead Block® 300-500 μm (nominal size range) microspheres in swine kidney (no iodine).

FIG. 5 is a graph illustrating the rate of weight gain in swine treated by BAE using radiopaque 102 microspheres.

FIG. 6 is a scatter plot showing the ulcer scores for radiopaque 102 microspheres in comparison to smaller (DC Bead LUMI® 40-90 μm (nominal)) and larger (DC Bead LUMI® 100-300 μm (nominal)) microspheres. The 40-90 size range has an iodine content of between 131 and 169 mg/ml and the 100-300 μm size range has an iodine content of between 122 to 162 mg/ml. The scatterplot gives the mean ulcer score and standard deviation for each microsphere population. Ulcer score: no ulcer=0, small (<=2 cm)=1, large (>2 cm)=2, full thickness ulceration=3

FIG. 7 is an alternative representation of the data of FIG. 6 in which “BAE bead” is the 102 microspheres of FIG. 6 (100-200 um) and ulcer scores are normalised.

FIG. 8 is a graph plotting weight gain for individual swine against fundal coverage. The data is derived from the cone beam CT scans of individual animals in example 4 (102 microspheres) where the fundal coverage is the extent of radiopacity within the fundus as a proportion of total fundal area.

### DETAILED DESCRIPTION

present disclosureAs previously noted, the present disclosure relates to compositions and methods useful in therapeutic embolisation and particularly in methods for bariatric arterial embolisation (BAE).

Therapeutic embolisation is a minimally invasive procedure in which a material is introduced into a blood vessel by the trans catheter route, in order to occlude the vessel and thus slow or stop blood flow leading to ischemia in the supplied tissue. This approach has been used for some time in the treatment of hyper-vascular tumours such as hepatocellular carcinoma, and for the treatment of benign growths such as uterine fibroids.

Recent pre-clinical observations have suggested that embolisation of blood vessels supplying the gastric fundus (known as bariatric arterial embolisation or BAE) may be useful in the control of weight gain, particularly for the treatment of obesity and associated squalae (Arepally et al 2007, Bawudun et al, 2012, Paxton et al 2013, Kipshidze et al 2013, Weiss et al 2014). These studies suggest that BAE leads, i.a., to a reduction in weight gain, a decrease in circulating ghrelin levels and a reduction in the numbers of ghrelin secreting cells in the fundus. U.S. Pat. No. 9,572,700, for example, describes BAE procedures using microspheres of 300-500 um size range (BeadBlock® 300-500 Biocompatibles UK Ltd) and suggests that smaller size ranges can result in mucosal necrosis of the fundus, gastric ulcers and off target embolisation for example, of the oesophagus, liver and/or spleen. However, Fu et al (2018) were unable to demonstrate a suppression of weight gain or a reduction in ghrelin expressing cells in pigs using microspheres of 300-500 μm nominal diameter.

Although the procedure holds promise, there have been persistent reports of adverse events such ulceration of the mucosal surface, e.g. of the gastric body and gastritis in animal models (Paxton et al 2014, and Weiss et al 2014). Thus BAE is a potentially useful approach to modulation of weight gain, obesity, and associated sequalae, however it is desirable to provide compositions and methods that result in effective embolisation of the gastric fundus but with an improved safety profile.

The inventors have identified that a key factor in the control of mucosal damage is the depth, within the vascular bed, at which the embolus occurs, and the presence of off-target embolisation of mucosal regions outside the fundus. The inventors have further identified that one cause of mucosal damage is the presence of microspheres within the submucosa itself, whilst when embolisation occurs only slightly more proximally to the catheter (i.e. in a direction away from the mucosa) this is effective at causing ischemia, but does not typically lead to long term or significant mucosal damage. On the other hand, it is believed that embolisation at a position that is too proximal, i.e. too far away from the mucosa, is of reduced efficacy because the embolic effects are reduced by the presence of collaterals within the stomach wall.

Microspheres are typically provided as populations of spheres having a spread of sizes, depending on the methods used to prepare them and the sizing techniques used, but the penetrability of the microspheres themselves is governed by a variety of factors. These include not only the size distribution, but also the compressibility (compressive modulus) of the spheres.

It is particularly useful to be able to visualise the microspheres in situ, because this enables the operator to determine where the microspheres are deposited in real time, and also identifies any off target embolisation, however, addition of e.g. radiopacifying components to the polymer, may alter the compressibility of the spheres and thus may influence their penetrability.

In a first aspect, the present disclosure therefore provides a composition comprising a population of polymeric microspheres having a native size distribution such that not more than 10% of the microspheres have a diameter less than 100 μm and not more than 10% of the microspheres have a diameter greater than 200 μm.

Native size is the size of the microspheres before injection. For water swellable polymers such as hydrogels, this is the size of fully hydrated microspheres in normal saline (10 mM phosphate; 500 mM NaCl; pH7.4).

Preferably the microspheres have a native size distribution such that not more than 5% of the microspheres have a diameter less than 100 μm; more preferably, and alternatively, not more than 5% of the microspheres have a diameter less than 120 μm.

Preferably the microspheres have a native size distribution such that not more than 5% of the microspheres have a diameter greater than 200 μm; more preferably, and alternatively, not more than 10% of the microspheres have a diameter greater than 185 μm.

In particularly preferred combinations, the microspheres have a native size distribution such that not more than 5% of the microspheres have a diameter less than 100 μm and not more than 5% of the microspheres have a diameter greater than 200 μm; more preferably the microspheres have a native size distribution such that not more than 5% of the microspheres have a diameter less than 120 μm and not more than 10% of the microspheres have a diameter greater than 185 μm.

The above native size distribution preferences are to be construed as alternative rather than additive.

The compressibility (compression modulus) of the microspheres affects the depth of penetration into the vascular bed. The more compressible the microsphere, the deeper within the vascular bed it penetrates for a given size. The measurement of the compression modulus of microspheres is described in Caine et al (2017) and also in Duran et al (2016). To the extent that the method of measurement laid out herein deviates from those methods, the presently described method should be followed; see Example 2 herein.

As referred to herein, mean compression modulus is the mean of at least 5 measurements taken from individual microspheres, although the skilled person will be aware that the more readings are taken, the more accurate will be the mean and so it is preferred that the modulus will be the mean of at least 25 measurements. Where the microsphere is a hydrogel, this should be measured when fully hydrated in normal saline.

The preferred modulus of the microspheres is at least 500 kP to 1000 kPa, and preferably at least 2,000 kPa more preferably at least 4000 kP and yet more preferably at least 5000 kP. Preferably the modulus does not exceed 50,000 kPa as such microspheres become more difficult to deliver, as their stiffness increases the tendency to cause blockages in catheters, although this also depends somewhat on catheter size. The modulus preferably does not exceed 30000 kP and more preferably does not exceed 25000 kPa

The preferred range of modus is 2,000 kP to 30,000 kP and more preferably 5000 kP to 25000 kPa.

Thus in one preferred aspect, the composition comprises a population of polymeric microspheres having a native size distribution such that not more than 10% of the microspheres have a diameter less than 100 μm and not more than 10% of the microspheres have a diameter greater than 200 μm; wherein the microspheres have a mean compression modulus of at least 1000 kPa.

Compression modulus may also be expressed as a relative term. Thus in microspheres preferably have a compressibility modulus of at least 5 times that of Bead Block® 300-500 microspheres. Bead Block® microspheres may be prepared according to WO04071495 Example 1, low AMPS version and sieved to 300-500 size range.

Preferably microspheres have a modulus of at least 10 times, more preferably at least 15 times yet more preferably at least 20 times and more preferably still, at least 25 times that of Bead Block® 300-500.

Preferably the microsphere will not have a compression modulus of more than 200 times that of BeadBlock® 300-500, preferably no more than 150 times more preferably no more than 125 times more preferably still no more than 110 times and yet more preferably not more than 100 times that of Bead Block® 300-500.

Preferably the microsphere the microspheres will have a compression modulus of 10 to 200 times that of BeadBlock® 300-500. More preferably 15 to 150, more preferably still 20 110 time and yet more preferably 25 to 110 or 25 to 100 times that of Bead Block® 300-500.

Thus in a further preferred aspect, the composition comprises a population of polymeric microspheres having a native size distribution such that not more than 10% of the microspheres have a diameter less than 100 μm and not more than 10% of the microspheres have a diameter greater than 200 μm; wherein the microspheres have a mean compression modulus of at least 5 times that of BeadBlock 300-500.

The depth, within the vascular bed, to which the microspheres penetrate is governed by a number of factors, including the native size of the microspheres and also their compressibility (compression modulus). As used herein the “penetration value” for a microsphere is the smallest diameter of the blood vessel at the point where a single microsphere lodges when blocking that vessel. This is determined in a swine kidney model, in which a population of microspheres is delivered to the renal artery to cause embolisation of the kidney vasculature (see for example Caine et al 2017). The penetration value is determined microscopically, following necropsy. Embolised kidneys are sectioned and stained and the smallest diameter of blood vessels embolised by a single microsphere are measured (many vessels are cut at an angle revealing an ellipse, the smallest diameter of the vessel is the smallest diameter of the ellipse). This is the penetration value of the microsphere (see also Example 3).

The above microspheres populations may have penetration characteristics as described herein below as per the second aspect.

In a second aspect, the present disclosure also provides a composition comprising a population of polymeric microspheres in which not more than 10% of the microspheres have a penetration value, in a swine kidney model, of less than 80 μm.

Preferably, not more than 5% of the microspheres have a penetration value of less than 80 μm, and alternatively and more preferably not more than 5% have a penetration value of less than 90 μm. Preferably not more than 10% of the microspheres have a penetration value of greater than 300 μm, more preferably not more than 5% of the microspheres have a penetration value of greater than 300 μm. Alternatively and still more preferably not more than 5% of the microspheres have a penetration value of greater than 250 μm. Preferably, in the population of microspheres, not more than 10% of the microspheres have a penetration value, in a swine kidney model, of less than 80 μm and not more than 10% of the microspheres have a penetration value of greater than 300 μm. More preferably, not more than 5% of the microspheres have a penetration value of less than 80 μm and not more than 5% of the microspheres have a penetration value of greater than 300 μm. Alternatively and still more preferably not more than 5% have a penetration value of less than 90 μm and not more than 5% of the microspheres have a penetration value of greater than 250 μm.

The above upper limit and lower limit penetration distribution preferences are to be construed as alternative rather than additive.

Such populations may have native size distributions and compressibility characteristics as described herein above in respect of the first aspect.

Preferably the polymer is a hydrophilic polymer, since such polymers are generally more biocompatible.

A hydrophilic polymer may be selected from the group consisting of: acrylic polymers, acrylamides, acetals, allyls, polyamides, polycarbonates, polyesters, polyethers, polyimides, polyolefins, polyphosphates, polyurethanes, styrenics, vinyls, polysaccharides, or combinations and/or copolymers thereof. Preferably the polymer comprises monomers selected from: vinyl alcohols, ethylene or propylene glycols, acrylates methacrylates, acrylamides or methacrylamides.

Preferred hydrophilic polymers include vinyl alcohol polymers such as polyvinylalcohol (PVA); acrylic polymers such as polyacrylic acids and salts, poly (alkylacrylates), such as poly(methylacrylates); poly alkyl(alkylacrylate)s, such as poly methylmethacrylates and polyethylmethacrylates; poly hydroxyalkyl(alkylacrylates) such as polyhydroxyethylmethacrylate; acrylamide polymers such as polyacrylamides, poly (alkylacrylamides), such as poly methacrylamides (hydroxyalkyl)acrylamides such as Tris-(hydroxymethy)methylacrylamaide; polyvinyl pyrrolidones, polyethylene glycol (PEG) polymers, such as PEG, PEG-acrylamides and diacrylamides, PEG-acrylates and diacrylates, PEG-methacylates and dimethacrylates; and PEG-methacrylamides and dimethacrylamides; celluloses such as carboxymethylcelluloses, hydroxyethylcelluloses; chitosans, alginates, gelatins, starches, or a combination or co-polymers comprising at least one of the foregoing. The polymers may be cross linked.

In a particular embodiment, the polymer comprises or is a polyhydroxylated polymer, i.e. a polymer that comprises repeating units bearing one or more pendant hydroxyls. Preferred polyhydroxylated polymers include those comprising poly(hydroxyalkylacrylates) and poly(hydroxyalkyl(alkylacrylates), particularly polyol esters of acrylates and alkylacrylates (e.g. methacylates), such as poly hydroxyethyl(methacrylate); poly(hydroxyalkylacrylamides) and poly(hydroxyalkylmethacrylamides), such as Tris(hydroxymethyl)methacrylamide; polymers comprising vinylalcohols such as poly(vinylalcohol) or (ethylene-vinylalcohol) copolymers; and polysaccharides such as starches, chitosans, glycogens, celluloses, such as methyl celluloses, alginates, and polysaccharide gums, such as carageenans, guars, xanthans, gellans, locus bean gums and gum arabics.

In a further embodiment, the hydrophilic polymer may be a poly carboxylated polymer i.e. a polymer that comprises repeating units bearing one or more pendant carboxyl groups. These polymers include, for example, poly acrylic acids poly alkylacrylic acids such as poly methacrylic acids and their co-polymers, particularly those with PVA. Such polymers may be in the form of their salts such as sodium or potassium salts.

Particularly preferred are polymers comprising PVA, such as homopolymers and co-polymers of poly vinyl alcohol (PVA), PEG polymers, such as PEG-acrylamides and diacrylamides, PEG-acrylates and diacrylates, PEG-methacylates and dimethacrylates; and PEG-methacrylamides and dimethacrylamides; and poly alkylacrylic acids such as poly methacrylic acids. Most preferred are polymers comprising PVA, such as homopolymers and co-polymers of poly vinyl alcohol

The polymers are preferably cross-linked polymers. Crosslinking may be covalent or non-covalent. Non-covalent includes, for example, physical crosslinking by entanglement of polymer chains, or by the presence of crystal regions. Ionic cross linking can occur where charged groups on the polymer are cross linked by polyvalent groups carrying the opposite charge. In some cases this can be through di or higher valent metal ions, such as calcium magnesium or barium, such as is the case with alginate polymers. Covalent cross linking can be achieved by any of the established methods to covalently link functional groups on different chains together. If achieved during the polymerisation stage this can be by incorporation of a bifunctional monomer. If post-polymerisation, then by a bifunctional species capable of reacting with functional groups on the polymer such as amine, hydroxyl or carboxyl groups or ethylenically unsaturated groups. The polymer may also carry pendant groups that themselves carry such cross linkable groups. For example, ethylenically unsaturated groups;

In a preferred embodiment, the polymer may be substituted by groups that are charged at pH 7.4. Such groups may carry positive or negative charges, which are able to reversibly bind compounds carrying the opposite charge at physiological pH (pH7.4). A variety of charged groups may be used, including sulphonate, phosphate, ammonium, phosphonium and carboxylate groups; carboxylate and sulphonate are preferred. In one embodiment of cross linked polymers, the charged group may be found on the cross linking moiety.

Particularly preferably the polymer is a hydrogel, that is to say, the polymer is water-swellable but water-insoluble. It may comprise greater than 50%, and preferably up to 98% water by weight, preferably 65 to 85% and more preferably 75 to 85% Polyhydroxylated or poly carboxylated polymers and preferably cross linked polyhydroxy polymers are preferred in this regard, due to their tendency to form such hydrogels.

In a particularly preferred embodiment the polymer is a cross linked poly vinyl alcohol polymer or co polymer in the form of a hydrogel. In one embodiment, such polymers may be crosslinked physically or covalently. Where the polymer is cross linked covalently, the polymer may comprise pendant groups (other than the —OH groups) bearing cross linkable groups, through which the polymer is cross linked, such as for example ethylenically unsaturated groups; or the polymer may be cross linked through a cross linker carrying two or more functional groups that react with the hydroxyl groups of the PVA backbone, such as aldehydes or acids

Particularly preferred are such polymers carrying a charged group as described above, particularly where the polymer comprises sulphonate or carboxylate groups (see for example WO2004/071495 and WO2017/037276)

One preferred type polymer is a polyvinyl alcohol macromer, having more than one ethylenically unsaturated pendant group per PVA molecule, formed by reaction of the PVA with ethylenically unsaturated monomers. The PVA macromer may be formed, for instance, by providing a PVA polymer, with pendant vinylic or acrylic groups. Pendant acrylic groups may be provided, for instance, by reacting acrylic or methacrylic acid with PVA to form ester linkages through some of the hydroxyl groups. Vinylic group-bearing compounds capable of being coupled to polyvinyl alcohol are described in, for instance, U.S. Pat. No. 4,978,713 and, preferably, U.S. Pat. Nos. 5,508,317 and 5,583,163. Thus the preferred macromer comprises a backbone of polyvinyl alcohol to which is coupled, to an (alk)acrylaminoalkyl moiety. One example of such a polymer comprises a PVA-N-acryloylaminoacetaldehyde (NAAADA) macromer, known as Nelfilcon-B or acrylamide-PVA.

In one preferred embodiment this macromer may be reacted with ethylenically unsaturated monomers optionally bearing a positive or negative charges, such as 2-acrylamido-2-methylpropane sulfonic acid (AMPS). Such polymers and methods of making them are described in WO04/071495, WO12/101455 and WO17/037276. DC Bead® is one such polymer microsphere.

Particularly preferably, microspheres may be imageable. This assists in visualisation during or post procedure. Imageability includes by ultrasound, X-Ray, magnetic resonance imaging, superparamagnetic resonance imaging, positron emission imaging (such as PET) or photon emission imaging (such as SPECT). Imageability is achieved by incorporating an imageable component, which is preferably incorporated throughout the microsphere. It is particularly preferred that such an agent is covalently attached to the polymer of the microsphere.

In a preferred embodiment the microsphere is imageable by X-ray. This can be achieved by incorporating a radiopacifying component into the polymer microsphere either covalently or non covalently. Examples of non covalently incorporated radiopacifying components include, for example particulate materials, such as barium salts (e.g. barium sulphate) (see, for example, Thanoo et al 1991), metals such as gold iron or tantalum, or iodinated oils such as Lipiodol®. However, in a more preferred approach, the polymer may comprise a covalently coupled radiopacifying component, such as iodine (e.g. WO2015/033092) or Bismuth (e.g. WO2018/093566), which is preferably coupled throughout the microsphere.

In one approach, the polymer microspheres comprise a covalently coupled group, such as a pendant group, comprising the radiopacifying component. Preferably, the covalently coupled pendant group is an iodinated group, such as an iodinated aromatic group, particularly a phenyl group. It will be understood by the person skilled in the art that the amount of iodine in the polymer may controlled by controlling the degree of coupling of the iodinated group to the polymer, for example, in PVA, the number of pendant groups in the polymer, or the number of iodines on the pendant group, for example. The iodine level may conveniently be expressed as amount of iodine (in mg) per ml of microspheres. Where the microspheres are water swellable, for example in hydrogels, such as cross linked PVAs, this refers to the amount of iodine per ml of fully hydrated beads, in normal saline as a packed volume (e.g., as quantified in a measuring cylinder). In the present disclosure, the microspheres have levels of iodine selected to provide appropriate radiopacity (or radiodensity) whilst ensuring that the compressability of the microspheres still provides the level of handling and penetration required and that the ease of catheter delivery and suspension characteristics are not unduly compromised.

Microsphere populations described herein may have levels of iodine in the polymer in the range 70-150 mg/mL, preferably 80-140 mg/mL, more preferably 85-120 mg/mL and particularly 90-110 mg/mL of settled microspheres. These levels have been found to provide good properties, especially for microspheres where the polymer is a cross linked PVA polymer or co-polymer as described herein.

Such groups may be coupled to the polymer backbone through a variety of chemistries, depending on the availability of functional groups on the polymer. For example, for polyhydroxylated polymers the pendant group may be coupled via an ether, ester or cyclic acetal linkage. Iodinated aromatic groups may be coupled to the polymer via a linker or directly through the coupling group. Suitable linkers include those having a chain of 1 to 6 atoms selected from C, N, S and O, between the aromatic group and the coupling group, provided that the chain contains no more than one atom selected from N, S and O; wherein C is optionally substituted by a group selected from ═O, —CH₃ and (—CH₃)₂, particularly ═O; wherein N is substituted by R¹, where R¹ is selected from H and C₁₋₄ alkyl, particularly H and methyl; and wherein S is an —SO₂— group. Within this linker S is less preferred. Suitable linkers include groups of the formula —(CH₂)ₚ—O—(CH₂)_{q}— wherein p and q are 0, 1 or 2, provided that p and q may not both be 0; —(CH₂)ₙNHC(O)— where n is 1 or 2; and C₁₋₆ alkylene; Preferable linkers are selected from methylene ethylene and propylene groups, methoxylene, ethoxylene, oxymethylene and oxyethylene groups, —(CH₂)ₙNHC(O)—, where n is 1 or 2; methylene, ethylene and propylene groups.

Where the polymer is, or comprises PVA (PVA polymers and co-polymers), the pendant group comprising the radiopacifying component (preferably an iodinated phenyl group) can be conveniently coupled to the polymer through a cyclic acetal group as described in WO2015/033092 and WO2015/03309. Thus in one particularly preferred embodiment, the microsphere comprises a cross linked poly vinyl alcohol polymer or co polymer in the form of a hydrogel as described above, wherein the PVA backbone additionally comprises an iodinated phenyl group coupled to the PVA backbone for example via a cyclic acetal linkage, and preferably coupled directly via the cyclic acetal.

Suitable iodinated phenyl groups are illustrated below:

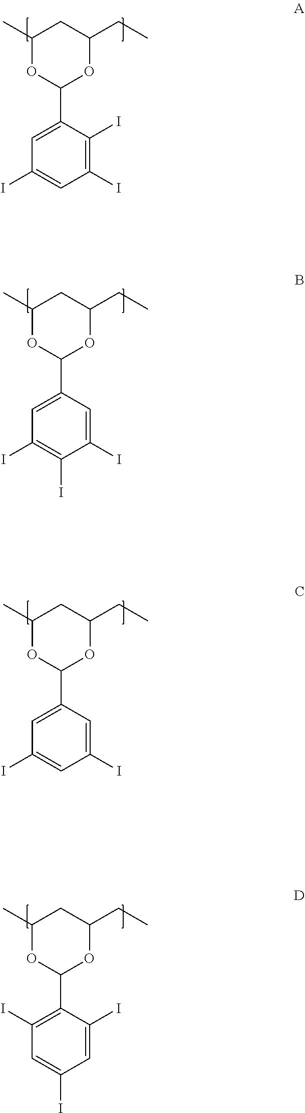

The most preferred pendant group is a group of the formula A:

Processes for preparing PVA polymers and co-polymers with such pendant groups are described in WO2015/033092 and WO2015/03309.

Thus in one particularly preferred approach, the polymer is a hydrogel, in the form of a cross linked PVA polymer or co-polymer, as described herein, comprising, throughout the polymer, a covalently attached iodinated group, such that the polymer comprises 70 to 150 mg/ml iodine.

In one approach, an effective amount of one or more pharmaceutical active agents can be included in the compositions. It may be desirable to deliver the active agent from the microspheres and so the microspheres may comprise such active agents, which may for example be bound to the polymer by ionic interaction or may be incorporated into the polymer.

In one advantageous embodiment, the microspheres of the present disclosure have a net charge such that charged pharmaceutical actives may be loaded into the microsphere e.g. by an ion exchange mechanism. As a result, the therapeutic agent is electrostatically held in the hydrogel and elutes from the hydrogel in electrolytic media, such as saline or in-vivo, e.g. in the blood or tissues, to provide a sustained release of drug over several hours, days or even weeks. In this embodiment it is particularly useful if the microspheres of the present disclosure have a net negative charge over a range of pH, including physiological conditions (pH7.4) such that positively charged drugs may be controllably and reproducibly loaded into the microsphere, and retained therein electrostatically, for subsequent prolonged elution from the hydrogel in-vivo. Such charges may be derived from ion exchange groups such as carboxyl or sulphonate groups attached to the polymer matrix. It will be understood that drugs without charge at physiological pHs may still be loaded into microspheres of the present disclosure and this may be particularly advantageous when rapid elution or a “burst effect” is desired, for example, immediately after embolisation or where their low solubility under physiological conditions determines their release profile rather than ionic interaction.

Examples of such compounds include those that suppress plasma ghrelin levels; such as somatostatin and somatostatin analogues e.g. octoreotide (typically as the acetate), amino acids, such as L-cyteine (McGavin et al 2015) or homones such as insulin (Saad et al 2002) and GLP-1

The populations of microspheres described herein will usually comprise at least 1000 microspheres and more typically will be provided in units of at least 25 or 50 μL of settled volume, preferably at least 100 μL, more preferably at least 250 μL settled volume of microspheres.

A third aspect the present disclosure provides a pharmaceutical composition comprising a population of microspheres as described herein and a pharmaceutically active agent wherein the therapeutic agent may be absorbed into the microsphere matrix. Such actives may be present in a pharmacologically effective amount in the population, i.e. the amount of active agent or microspheres required to obtain the desired effect from the population of microspheres. Such compositions typically comprise the presently described microspheres and a pharmaceutically acceptable diluent or carrier, typically an aqueous diluent or carrier. The aqueous diluent or carrier is preferably sterile, and may, for example be sterile water for injection or a saline solution, preferably buffered at an appropriate pH, for example between 7 and 8, for example pH 7.4±0.2. Water for injection or normal saline are typical. The diluent or carrier will typically be suitable for injection or infusion, and so, for example, will typically be free of pyrogens.

Pharmaceutical compositions may also comprise additional components such as contrast agents, (either ionic or non ionic and/or oily contrast agents such as ethiodised poppy seed oil (Lipiodol®). Suitable non-ionic contrast agents include iopamidol, iodixanol, iohexol, iopromide, iobtiridol, iomeprol, iopentol, iopamiron, ioxilan, iotrolan, iotrol and ioversol. Ionic contrast agents may also be used, but are not preferred, especially in combination with drug loaded microspheres, where the polymer carries an ionic charge, since high ionic concentrations favour disassociation of ionic drugs from the matrix. Ionic contrast agents include diatrizoate, metrizoate and ioxaglate.

Alternatively, the radiopaque hydrogel microspheres of the present disclosure may be provided in a dried form. Where microspheres or other radiopaque polymer products are provided dry, it is advantageous to incorporate a pharmaceutically acceptable water soluble poly-ol into the polymer before drying. This is particularly advantageous for hydrogels as it protects the hydrogel matrix in the absence of water. Useful poly-ols are freely water soluble sugars (mono or di saccharides), including glucose, sucrose, trehalose, mannitol and sorbitol.

The microspheres may be dried by any process that is recognised in the art, however, drying under vacuum, such as by freeze drying (lyophilisation) is advantageous as it allows the microspheres to be stored dry and under reduced pressure. This approach leads to improved rehydration as discussed in WO07147902 (which is incorporated herein by reference). Typically, the pressure under which the dried microspheres are stored is less than 1 mBar (gauge).

Delivery of the present composition of microspheres to the gastric fundus induces weight loss or reduces the rate of weight gain in a subject. Delivery is typically by the transcatheter route. Suitable subjects include mammalian subjects, most particularly in a human subjects, however the approach may also be used in other mammalian species and might, for example, also be used to induce weight loss or reduce the rate of weight gain in mammalian companion, or other, animals such as cats, dogs and horses.

In a fourth aspect, the present disclosure therefore provides a method of inducing weight loss or of slowing weight gain in a subject comprising delivering to the capillary bed of the gastric fundus of the subject, an effective amount of a population of microspheres as described herein. Such compositions may be delivered in the form of a pharmaceutical composition as described herein.

The effective amount of microspheres is the amount necessary to provide a measurable improvement in the indication to be treated. this volume depends upon the subject to be treated but for larger mammals such as humans is typically in the range 50 uL to 1000 uL to 1600 uL, and preferably 100 to 800 uL, and more preferably 150 to 750 uL, measured as packed microsphere volume.

Treatment of a condition in which the subject is in need of reduction in body weight or a reduction in the rate of weight gain, such as for example in obesity, is also expected to lead to a relief of comorbidities of the condition, or to a reduction in the risk of such conditions. Such conditions include chronic conditions such as insulin resistance, type 2 diabetes mellitus, hypertension, dyslipidemia, cardiovascular disease, sleep apnea, gallbladder disease, hyperuricemia, gout, and osteoarthritis, as well as acute conditions such as stroke. Thus in further aspects, the present disclosure therefore also provides methods for the treatment of these comorbidities also. Since BAE is known to lead to lowering of ghrelin levels, a reduction in the numbers of ghrelin secreting cells in the fundus and a reduction in hunger, the present disclosure also provides, in yet further aspects, methods of lowering ghrelin levels in the blood of a subject, of reducing the number of ghrelin secreting cells in the gastric fundus of a subject and methods for reducing hunger in a subject.

In a fifth aspect is provided the use of a composition comprising a population of microspheres according to any of the aspects herein in the manufacture of a medicament for the treatment or prevention of any the conditions recited herein. In a further aspect is provided a composition comprising a population of microspheres according to any of the aspects herein, for use in any of the methods of treatment described herein. In each case the method includes delivering to the capillary bed of the gastric fundus of the subject, the compositions described.

The present disclosure will now be described further by way of the following non limiting examples with reference to the figures. These are provided for the purpose of illustration only and other examples falling within the scope of the claims will occur to those skilled in the art in the light of these. All literature references cited herein are incorporated by reference.

### EXPERIMENTAL EXAMPLES

### Example 1. Preparation of Microspheres

Cross-linked hydrogel microspheres were prepared according to Example 1 (High AMPS version) of WO 2004/071495. The process was terminated after the step in which the product was vacuum dried to remove residual solvents and microspheres were then sieved to provide appropriate size ranges. Sieves of 500 μm, 425 μm, 355 μm, 323 μm, 250 μm, 212 μm and 160 μm we re used sequentially and microspheres were collected from the following sieves to provide the samples used: 355-425 μm (“304”), 250-323 μm (“203”) and 160-212 μm (“102”). Beads were stored dry and when needed, acetalized with 2,3,5-triiodo benzaldehyde according to the method described in WO2015/033093 to provide radiopaque, iodinated microspheres.

Briefly, 1 g of dry microspheres and the appropriate amount of aldehyde (see table 1 below) were placed in a vessel purged with nitrogen. 30 ml anhydrous DMSO were added under a nitrogen blanket and stirred to keep the beads in suspension. The suspension was warmed to 50° C. and 2.2 ml of methane sulphonic acid was added slowly. The reaction slurry was stirred at 50° C. for 22 hours, while the consumption of aldehyde was monitored by HPLC. The reaction slurry was then allowed to settle and the reaction mixture was removed by aspiration and the microspheres washed with 30 ml DMSO/0.5% NaCl ×5, followed by 50 ml of 0.9% NaCl, ×5. Washes were carried out at 50° C. 1.5 ml samples of the resultant microspheres were then stored in 5 ml of phosphate buffered saline.

TABLE 1

| Samples | Sieve size | TIBA | mg/ml I |
|---|---|---|---|
| 102 | 160-212 μm | 0.9 | 129 |
| | | 0.6 | 84 |
| | | 0.75 | 95 |
| | | 1.4 | 158 |
| | | 1.8 | 158 |
| 203 | 250-323 | 0.53 | 49.9 |
| | | 0.66 | 68.1 |
| 304 | 355-425 | 0.8 | 113 |
| | | 1 | 140 |
| | | 1.2 | 146 |
| | | 0.53 | 67.5 |
| | | 0.66 | 85.3 |

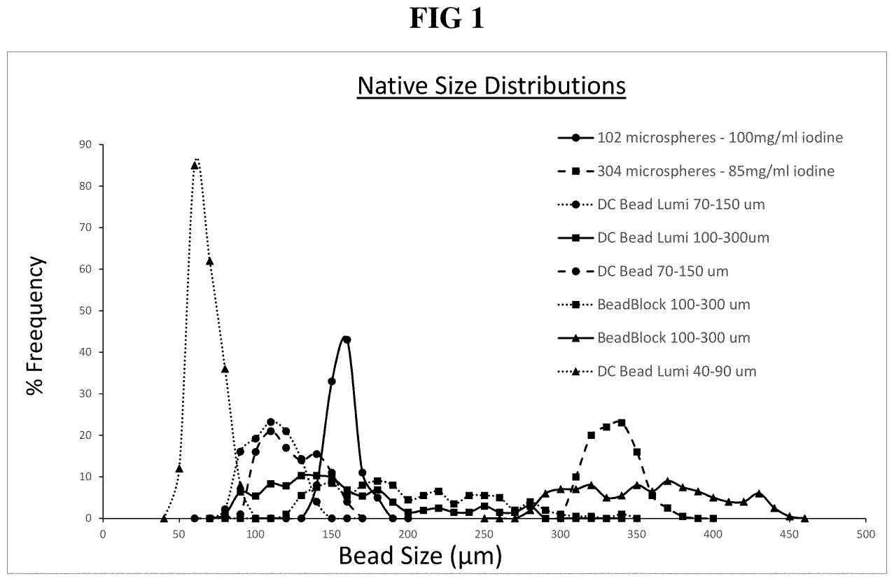

Actual microsphere size ranges were determined by measuring the diameter of approximately 200 individual random microspheres under a microscope. The results are shown in FIG. 1 in comparison with other commercially available cross linked PVA hydrogel based microspheres.

### Example 2. Measurement of Elastic Compressive Modulus (ECM) of Microspheres

The elastic compressive modulus (ECM) of microspheres may be measured according to the protocol outlined in Cain et al (2018) and Duran et al (2016). Caine et al (2018) also provides a table of compression modulus values for a variety of commercial microspheres. Briefly, ECM is determined using a UNHT Bioindentor system (Anton Paar, Switzerland) operated by the proprietary indentation software, with a force range of 0.01-20 mN and displacement range 1 nm to 100 μm. A sample of microspheres was dispersed in a dish and submerged in normal saline. Individual microspheres were selected using the optical microscope on the instrument and their diameters measured to the nearest 1 μm (at 5× Magnification). Individual microspheres were compressed at 50 μm/min, a 5 s pause was applied and then the sample was unloaded at 50 μm/min. Acquisition was set at 20 Hz. Elastic modulus of each bead was calculated from the loading curve, applying linear elastic Hertzian contact mechanics for the case of a sphere compressed between two flat surfaces and reported as the arithmetic mean of n=5 replicates in the 10-15% individual bead diameter compression range.

The results obtained for samples of experimental and commercial microspheres is given in Table 2.

TABLE 2

| | | Elastic | | | |
|---|---|---|---|---|---|
| | | Modulus | | | |
| Microsphere | Iodine | @ 15-20% | Std. | | |
| type | content | Comp | dev. | Relative | n |
| Bead Block ® | 0 | 242 | 57.0 | 1.0 | 5 |
| 300-500 μm | | | | | |
| DC Bead ® | 0 | 179 | 55.9 | 0.7 | 5 |
| 70-150 μm | | | | | |
| DC Bead | 150 | 28315 | 7625.1 | 117.1 | 5 |
| LUMI ® | | | | | |
| 70-150 μm | | | | | |
| 102-1 | 83 | 7548 | 294.6 | 31.2 | 5 |
| 102-2 | 92 | 9738 | 448.4 | 40.3 | 5 |
| 102-3 | 107 | 13366 | 3975.9 | 55.3 | 5 |
| 102-4 | 121 | 21240 | 5757.7 | 87.9 | 5 |
| 304-1 | 113 | 14388 | 1958.8 | 59.5 | 4 |

Bead Block® and DC Bead® are both crosslinked PVA microspheres prepared by crosslinking a PVA-N-acryloyl-aminoacetaldehyde dimethylacetal (NAADA) macromer with 2-acrylamido-2-methylpropanesulfonic acid as described in WO2004/071495. DC Bead LUMI® is prepared as per DC Bead® and substituted by iodinated phenyl groups as described in WO2015/033092.

### Example 3. In Vivo Renal Embolisation Procedure

Renal arteries of female Yorkshire swine of weight approximately 30 kg are embolised according to the following procedure:

The femoral artery is cannulated using an ultrasound-guided Seldinger technique. Through the needle of the micropuncture set, a wire is advanced into the abdominal aorta. Next, the needle is removed and a 5-6 Fr. vascular sheath is placed in the femoral artery. IV heparin may be administered at 5,000 IU and may be repeated, if needed, after several hours. A guide catheter is advanced over a wire into the aorta under X-ray fluoroscopy. Angiographic evaluation with iodinated contrast agents of the renal arteries is performed and an artery is selected under fluoroscopic guidance for embolisation. Nitroprusside (100 mg) may be injected intra-arterially to prevent vasospasm during selective artery catheterization.

Embolic microspheres are then injected into the selected vessel(s) using a 2.8 Fr Renegade® Hi-Flo microcatheter (Boston Scientific) using a gradual embolisation technique wherein small aliquots are beads are intermittently delivered allowing for the blood flow to carry the beads into the kidney before the next aliquot is administered. The animal is then humanely euthanized by saturated overdose barbiturate-based euthanasia and the kidneys harvested.

Serial sections of the kidney are taken in the ideal sectioning plane from the superior, inferior, and lateral poles encompassing the collecting duct, medulla, and cortex of the kidney and stained with haematoxylin and eosin. The sections are then digitally scanned and the furthest penetration of the microspheres evaluated.

Where a single microsphere is noted to have occluded a vessel, the diameter of the occluded vessels is measured as the internal diameter of the vessel lumen (for transversal vascular section) at that point, or as the smallest axis of the ellipse, for oblique sections. In case of longitudinal section, the vessel diameter was measured at the level of the largest microsphere. At least 140 vessel diameters per kidney are analysed.

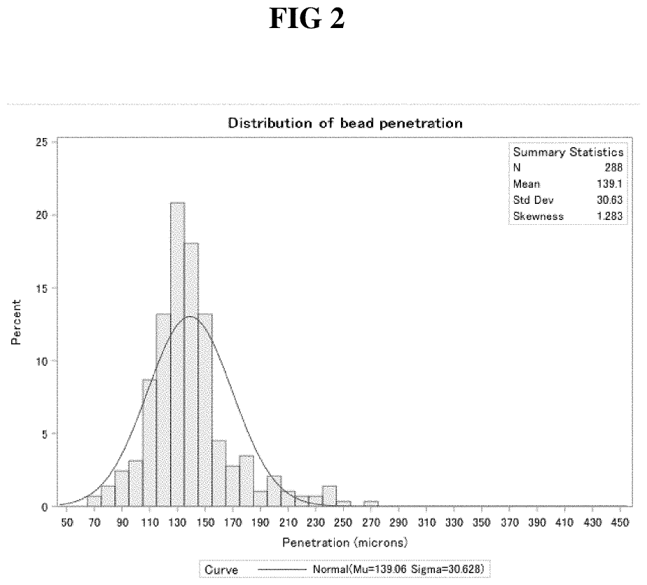

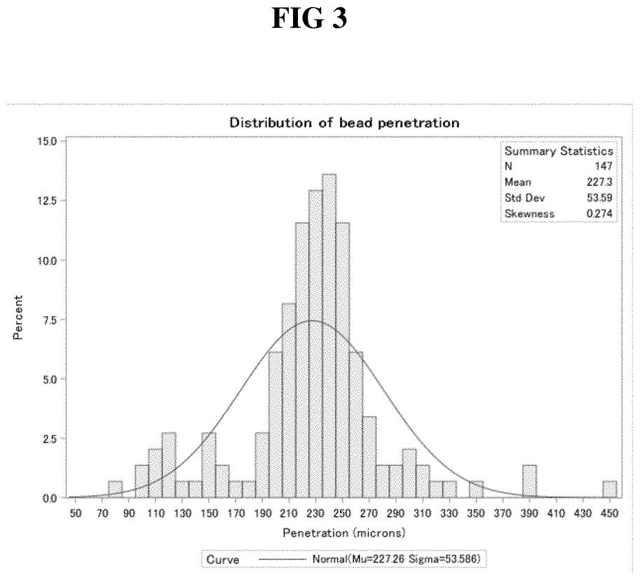

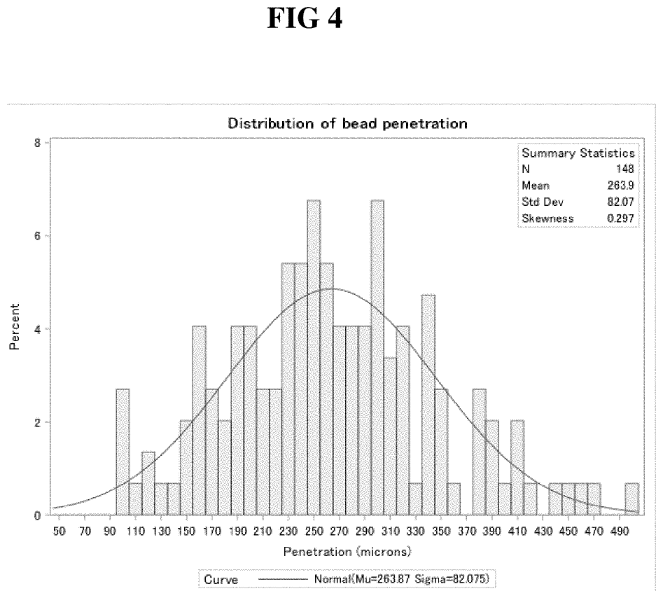

FIGS. 2, 3 and 4 show the penetration data for sample microsphere preparations of 102 (129 mg/ml iodine), 304 (113 mg/ml iodine) and for a commercially available preparation—BeadBlock® 300-500 μm (Biocompatibles UK Ltd)

### Example 4: Embolisation of Porcine Gastric Fundus

Radiopaque 102 microspheres (95 mg/ml iodine) were infused into the left gastroepiploic artery and right gastric artery of healthy, growing swine (˜23 kg). These two arteries supply the fundus. The microspheres were delivered diluted 1:10 in non ionic contrast. Three control swine underwent a sham procedure with saline infusion.

All swine were administered 40 mg of oral omeprazole daily from 3 days before to 28 days after BAE or the sham procedure as a gastroprotective agent to prevent ulceration as administered in previous trials.

Fasted swine were sedated with ketamine (100 mg/mL), xylazine, and telazol at 1 mL per 25 kg intramuscularly and induced with propofol to effect intravenously (˜4 mg/kg). General anesthesia was maintained with 1-2% isoflurane (Baxter Healthcare Corp., Deerfield, Ill.). Swine were intubated and mechanically ventilated.

Femoral arterial access was obtained percutaneously under ultrasound guidance (Zonare Medical Systems, Inc., Mountain View, Calif.) followed by introducer sheath (5 Fr) placement. Under x-ray fluoroscopic guidance (Axiom Artis Zee, Forchheim, Germany), a 5 Fr angiographic guide catheter (Flexion Axis, Surefire Medical, Westminster, Colo.) was advanced over a 0.035-inch Bentson guidewire (Cook Medical, Bloomington Ind.) into the abdominal aorta to select the celiac axis. A pre-embolisation celiac digital subtraction angiogram (DSA) with iohexol injection at 4 mL/sec for 5 seconds was then obtained to map the vessels feeding the gastric fundus. A microcatheter (Renegade™) was then advanced over a 0.016-inch Fathom guidewire (Boston Scientific Corp., Marlborough, Mass.) into the fundal branches of the gastric artery. A DSA of the selected vessel was acquired with gentle hand puff of 50% of iohexol to confirm the sub selection of the target artery. One hundred micrograms of nitroprusside was then delivered into that vessel as a muscle relaxant and to prevent spasm during microcatheter deployment. The artery was then embolized with the microspheres until five beats of stasis was achieved after this point the second artery was selected using hand-puff DSA and embolized to five beat status. Intermediate single shots were acquired to document the location of the embolic beads. Hand-puff DSA was then acquired to confirm the embolisation of the target arteries. If residual flow greater than 5 beat stasis was observed, further embolic was administered. Post-embolisation CBCTs were acquired to confirm the success of embolisation. The microcatheter was removed, flushed with saline, and repositioned before embolisation of the next arterial branch.

Weight was measured at baseline and at weeks 1-8 post-embolisation. Celiac digital subtraction angiographs (DSA) were acquired prior to and immediately after embolisation, and at 8 weeks post embolisation. Cone beam CT (CBCT) images of the stomachs were acquired immediately after embolisation and at 8 weeks prior to sacrifice. Endoscopy of the stomach was performed at approximately 1 week after embolisation to assess the effect of the microspheres on the stomach mucosa with a standard adult gastroscope (Pentax, Denver, Colo.).

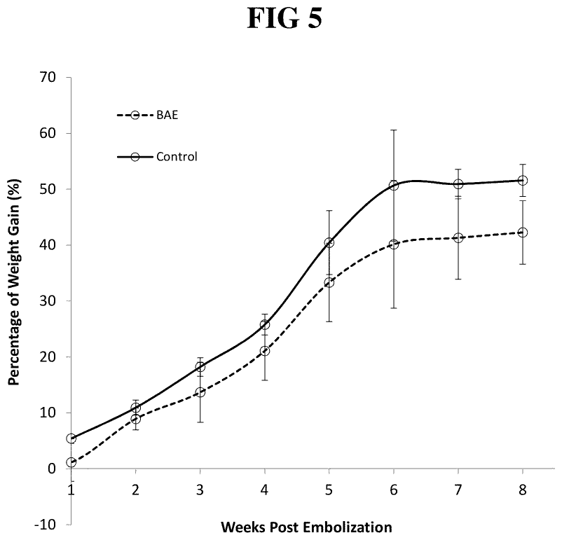

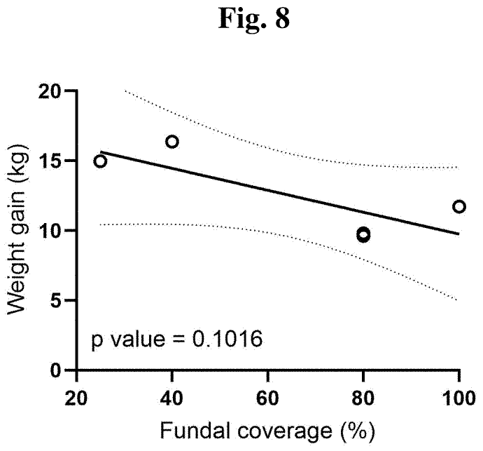

Radiopaque microspheres were visualized on CBCT images up to 8 weeks post embolisation. Week 1 endoscopic evaluation revealed that all bariatric arterial embolisation animals developed small, superficial, mucosal ulcers in the gastric fundus or body which were healed by week 8, while control animals were absent of ulcers. A significant decrease in percentage of weight gain was noted in bariatric arterial embolisation animals as compared to controls (bariatric arterial embolisation vs. control: 42.3%±5.7 vs. 51.6%±2.9, p<0.001). Body mass progress is shown in FIG. 5, showing that the 100-200 μm microspheres were effective at reducing weight gain in a swine model. FIG. 8 illustrates the relationship between fundal coverage and weight gain. The data is derived from the cone beam CT scans of individual animals. Fundal coverage being the extent of radiopacity within the fundus as a proportion of total fundal area. This represents the degree of embolization within the fundus region.

Table 3 shows the incidence of ulceration in animals at 1 week.

TABLE 3

| | Microsphere | | | |
|---|---|---|---|---|
| Animal | settled volume | | Ulcer | |
| ID | delivered (ml) | Ulcer | score | Comment |
| Control 1 | 0 | No | 0 | — |
| Control 2 | 0 | No | 0 | — |
| Control 3 | 0 | No | 0 | — |
| TEST 1 | 140 | Yes | 1 | Small at lesser curvature |
| | | | | to fundus |
| TEST2 | 230 | Yes | 1 | Small at lesser curvature |
| | | | | to fundus |
| TEST 4 | 250 | Yes | 2 | Small to medium, but |
| | | | | smaller than LUMI26 |
| TEST 5 | 330 | Yes | 2 | Huge ulcer at lesser to |
| | | | | fundus, not deep, food in |
| | | | | stomach |
| TEST 6 | 315 | Yes | 1 | Tiny ulcer |
| TEST 7 | 310 | Yes | 1 | Two small ulcers: one |
| | | | | with fibrin cap, the other |
| | | | | looks like a tiny spot of |
| | | | | discoloration |

* Test animal 3 died within 24 hrs of the operation for reasons not related to the embolisation. In Test animal 5 a large ulcer was seen. It was not clear whether this was related to the treatment. The animal was euthanised 2 weeks after embolisation due to non treatment related issues.

### Example 5 BAE with Alternatively Sized Microspheres

Example 4 was repeated using commercially available radiopaque microspheres (DC Bead LUMI® 40-90 μm nominal size and 100-300 μm nominal size—Biocompatibles UK.). For each of these products, greater than 10% of microspheres were smaller than 100 μm.

Table 4 below shows the incidence of ulceration in animals.

TABLE 4

| | | Endoscopy | Microspheres | | | |
|---|---|---|---|---|---|---|
| Animal | | (days | set. vol. | | Ulcer | |
| ID | Identifier | post op.) | (ul) | Ulcer | score | Comment |
| Control 1 | Control | 14 d | 0 | No | 0 | — |
| Control 2 | Control | 6 d | 0 | No | 0 | — |
| Control 3 | Control | 7 d | 0 | No | 0 | — |
| Control 4 | Control | 6 d | 0 | No | 0 | — |
| Test 1 | S1 | 14 d | 190 | Yes | 1 | Small “tiny” <1 cm ulcer |
| | | | | | | at greater curvature |
| Test 2 | S1 | 6 d | 310 | Yes | 3 | Largest ulcer extending from |
| | | | | | | the fundus to almost the antrum. |
| Test 3 | S1 | 6 d | 200 | Yes | 2 | Ulceration much more in |
| | | | | | | lesser curvature |
| Test 4 | S1 | 7 d | 200 | Yes | 2 | >5 cm ulcer at lesser curvature |
| | | | | | | extending into fundus |
| Test 5 | S2 | 13 d | 520 | Yes | 1 | 2 cm superficial ulcer with |
| | | | | | | overlying exudate at the lesser |
| | | | | | | curvature/fundus. |
| Test 6 | S2 | 7 d | 600 | Yes | 2 | Large superficial healing ulcer |
| | | | | | | in the lesser curvature of the |
| | | | | | | stomach, not completely healed |
| Test 7 | S2 | 6 d | 230 | Yes | 2 | Lesser, greater and fundus |
| | | | | | | ulcers. Superficial. |
| Test 8 | S2 | 7 d | 230 | Yes | 1 | Small, shallow superficial |
| | | | | | | 1.5 cm lesser curvature |
| Test 9 | L2 | 13 d | 600 | Yes | 3 | Thin, 4 cm ulcer, shallow and |
| | | | | | | superficial |
| Test 10 | L2 | 7 d | 270 | Yes | 3 | Larger, deeper ulcer, healing at |
| | | | | | | edges, centered in lesser curvature |
| | | | | | | and extending to fundus |
| Test 11 | L2 | 6 d | 430 | Yes | 2 | Extensive ulcer from fundus to |
| | | | | | | lesser curvature. More than |
| | | | | | | superficial, not penetrating |
| Test 12 | L2 | 6 d | 230 | Yes | 2 | Large healing ulcer along lesser |
| | | | | | | curvature with exudate |

Ulcer score: no ulcer = 0, small (<= 2 cm) = 1, large (>2 cm) = 2, full thickness ulceration = 3

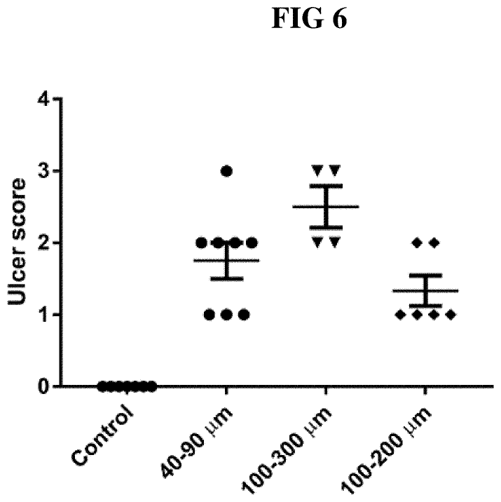

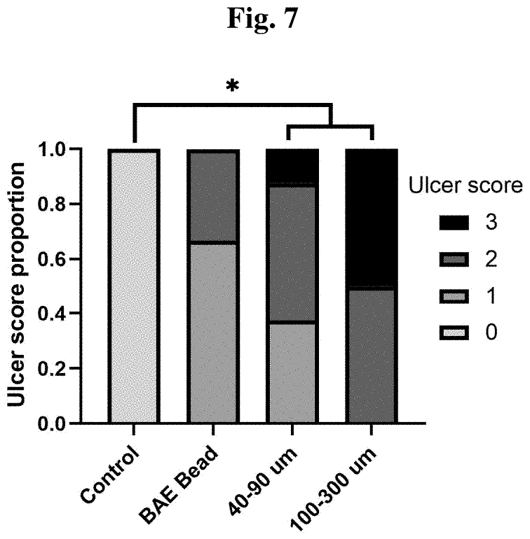

S1 animals were treated with DC Bead LUMI® 40-90 μm microspheres by delivery to one gastric artery. S2 animals were treated by delivery of DC BeadLUMI® 40-90 μm by delivery to two gastric arteries. L2 animals were treated by delivery of DC Bead LUMI® 100-300 μm microspheres to two gastric arteries. FIGS. 6 and 7 illustrate the level of ulceration seen in following BAE using the three microsphere types.

### REFERENCES

Arepally et al (2007) Radiology, 244:138-143
Bawudun et al (2012) Cardiovasc. Intervent. Radiol. 35:1460-1466.
Caine et al (2017) Journal of the Mechanical Behavior of Biomedical Materials 78: 46-55.
Duran et al (2016) Theranostics 6 (1): 28-39
Fu et al (2018) Radiology. 289(1):83-89.
Kipshidze et al (2013) Presented at the 62nd Annual Scientific Meeting of the American College of Cardiology; San Francisco, Calif. Mar. 10, 2013.
McGavin et al (2015) International Journal of Obesity volume 39, pages 447-455.
Paxton et al (2013) Radiology 266: 471-479.
Paxton et al (2014) J. Vasc. Interv. Radiol. 25: 455-461.
Saad et al (2002) J. Clin. Endocrinol. Metab. 87: 3997-4000.
Thanoo et al (1991) J. App. Biomaterials, 2: 67-72.
Weiss et al (2014) Presented at the 30th Annual Scientific Meeting of the European Society of Interventional Radiology; Glasgow, UK. September 13-17.

1. A composition comprising a population of polymeric microspheres comprising a polymer and having a native size distribution in which not more than 10% of the microspheres have a diameter of less than 120 μm and not more than 10% of the microspheres have a diameter greater than 200 μm.

2. A composition according to claim 1, wherein the microspheres have a mean compression modulus of greater than 1000 kPa.

3. A composition according to claim 1, wherein the microspheres have a mean compression modulus of at least 5 times that of Bead Block® 300-500.

4. A composition according to claim 1, wherein the microspheres have a native size distribution in which not more than 5% of the microspheres have a diameter less than 100 μm and not more than 5% of the microspheres have a diameter greater than 200 μm.

5. A composition according to claim 1, wherein the microspheres have a native size distribution such that not more than 5% of the microspheres have a diameter less than 120 μm and not more than 10% of the microspheres have a diameter greater than 185 μm.

6. A composition according to claim 1, wherein not more than 10% of the microspheres have a penetration value, in a swine kidney model, of less than 80 μm.

7. A composition according to claim 1, wherein not more than 10% of the microspheres have a penetration value of greater than 300 μm.

8. A composition according to claim 1, wherein not more than 5% of the microspheres have a penetration value of less than 80 μm and not more than 5% of the microspheres have a penetration value of greater than 300 μm.

9. A composition according to claim 1, wherein not more than 5% have a penetration value of less than 90 μm and not more than 5% of the microspheres have a penetration value of greater than 250 μm.

10. A composition comprising a population of polymeric microspheres which comprise a polymer and in which not more than 10% of the microspheres have a penetration value, in a swine kidney model, of less than 80 μm.

11. A composition according to claim 10, wherein not more than 10% of the microspheres have a penetration value of greater than 300 μm.

12. A composition according to claim 10, wherein not more than 5% of the microspheres have a penetration value of less than 80 μm and not more than 5% of the microspheres have a penetration value of greater than 300 μm.

13. A composition according to claim 10, wherein not more than 5% have a penetration value of less than 90 μm and not more than 5% of the microspheres have a penetration value of greater than 250 μm.

14. A composition according to claim 10, wherein the microspheres have a native size distribution in which not more than 10% of the microspheres have a diameter of less than 120 μm and not more than 10% of the microspheres have a diameter greater than 200 μm.

15. A composition according to claim 10, wherein the microspheres have a native size distribution such that not more than 5% of the microspheres have a diameter less than 120 μm and not more than 10% of the microspheres have a diameter greater than 185 μm.

16. A composition according to claim 10, wherein the microspheres have a mean compression modulus of greater than 1000 kPa.

17. A composition according to claim 10, wherein the microspheres have a mean compression modulus of at least 10 times that of Beadblock® 300-500.

18. A composition according to claim 1, wherein the polymer is a hydrogel.

19. A composition according to claim 1, wherein the polymer comprises poly vinyl alcohol.

20. A composition according to claim 1, wherein the polymer is imageable.

21. A composition according to claim 1, wherein the polymer is radiopaque.

22. A composition according to claim 1, wherein the polymer comprises between 70 and 150 mg of iodine per mL of settled microspheres covalently bound to the polymer, preferably 85-120 mg/mL and particularly 90-110 mg/mL of settled microspheres.

23. A pharmaceutical composition comprising a population of polymeric microspheres according to claim 1, and a pharmaceutically acceptable diluent.

24. A method of inducing weight loss or of slowing weight gain in a subject in need thereof, comprising delivering to the capillary bed of the gastric fundus of the subject, an effective amount of a composition comprising a population of microspheres according to claim 1.

25. A method for the treatment of obesity in a subject in need thereof, comprising delivering to the capillary bed of the gastric fundus of the subject, an effective amount of a composition comprising a population of microspheres according to claim 1.

26. A method according to claim 24, wherein the microspheres are delivered by the transcatheter route.

27. A composition according to claim 1 for use in a method of inducing weight loss or of slowing weight gain in a subject in need thereof.
